(19) United States
(12) Patent Application Publication
(10) Pub. No.: US 2017/0218398 A1
(43) Pub. Date: Aug. 3, 2017

(54) METHOD TO SELECTIVELY TARGET CANCEROUS CELLS FOR GENETIC MANIPULATION

(71) Applicants: Markus Alexander Brown, Bethesda, MD (US); Reinhard Ebner, Gaithersburg, MD (US)

(72) Inventors: Markus Alexander Brown, Bethesda, MD (US); Reinhard Ebner, Gaithersburg, MD (US)

(21) Appl. No.: 15/419,599

(22) Filed: Jan. 30, 2017

Related U.S. Application Data
(60) Provisional application No. 62/289,231, filed on Jan. 30, 2016.

(51) Int. Cl. C12N 15/86; C12N 9/22; C12N 15/90

(52) U.S. Cl. CPC C12N 15/86; C12N 15/907; C12N 9/22; C12N 2840/007

(57) ABSTRACT

Methods and compositions for selectively targeting cancerous cells for genetic manipulation based on cancer specific sequence motifs (CSSMs), which are formed as a result of chromosomal rearrangement, and cells produced by said methods.

### CROSS-REFERENCE TO RELATED APPLICATIONS

This application claims the benefit of U.S. Provisional Patent Application Ser. No. 62/289,231, filed on Jan. 30, 2016, the entire contents of which are hereby incorporated by reference.

### TECHNICAL FIELD

This invention relates to methods of selectively targeting cancerous cells for genetic manipulation by cancer specific sequence motifs (CSSMs), and cells produced by said methods.

### BACKGROUND ART

Cancer is one of the leading causes of morbidity and mortality worldwide, responsible for approximately 8.2 million deaths each year, an estimated 13% of all deaths worldwide¹. In the United States, an estimated 595,690 people will die from cancer in 2016 and the most common cancers are projected to be of the breast, lung and bronchus, prostate, and colon and rectum². The national expenditure for cancer care in the United States reached approximately $125 billion in 2010, and by 2020, is anticipated to approach $156 billion³. Cancer is a severe societal and economic burden and it is of vast importance to develop an effective method to treat cancer to reduce the suffering of the human race.

Conventional cancer treatment has consisted of chemoradiotherapy and surgery, though we are witnessing the emergence of a new era of precision medicine. Newly evolving approaches such as virotherapy, and molecularly targeted therapies, including small molecule inhibitor-based therapies and immunotherapy are becoming increasingly popular and effective⁴⁻⁶. Advances in virotherapy, immunotherapy and small molecule inhibitor-based therapies are particularly exciting as they do not involve invasive procedures, an advantage over surgery, and can be used in patients not fit for intensive chemotherapy⁷. Unfortunately, progress in molecularly targeted therapies, which depend on high specificity, has been hampered due to tumor heterogeneity⁸. Tumor heterogeneity refers to the distinct genotypes and phenotypes found between tumors in different patients as well as between different cells within the same tumor. These are referred to as inter-tumor heterogeneity and intra-tumor heterogeneity, respectively. This genetic variance is responsible for differential treatment response and represents one of the major hurdles that has yet to be overcome in cancer biology^{9,10}.

Genome editing is a powerful genetic tool used to manipulate cellular behavior by altering the genes within the genome. Though past use of this technology has primarily focused on cell culture experiments to elucidate gene function, genome editing technologies are being prepared for use in a therapeutic context. Current applications of genome editing involve functional gene knockouts and the repair of mutated genes in monogenic disease. Successful applications of gene therapy in cell culture and mouse models include editing of the IL2RG gene in X-linked Severe Combined Immunodeficiency (SCID-X1), the β-globin gene in Sickle Cell Disease (SCD), regions of the Dystrophin gene in Duchenne Muscular Dystrophy (DMD), and the cystic fibrosis transmembrane conductance receptor (CFTR) gene in cystic fibrosis¹¹⁻¹⁴. Clinical trials will presumably follow once sufficient efficacy for each technique has been proven. One clinical trial has already been performed using ZFN-guided, ex vivo editing of CD4+ T cells to generate CCR5Δ32 mutants resistant to HIV-1 infection, which were reintroduced into patients following genome editing¹⁵. This landmark study demonstrated the feasibility of targeted genome editing for the treatment of disease. The largest challenges faced by genome editing strategies are the efficiency of gene correction and the delivery of the nucleases, and donor DNA when applicable, to the target cell population¹⁶. Genome editing has not been applied to cancer treatment and has remained outside the realm of possibility due to high genetic complexity and the aforementioned challenges.

### SUMMARY

### Technical Problems

The foremost technical problem with treating cancer is the formidable task of individuating between cancerous and non-cancerous cells. Successful differentiation allows for targeted approaches, which attack the cancer while leaving healthy cells unperturbed. Approaches exist which preferentially target cancerous cells, such as treatment with the small molecule inhibitor, imatinib¹⁷. This drug selectively inhibits a handful of tyrosine kinases, which is particularly effective in treating Philadelphia chromosome-positive chronic myelogenous leukemia (CML) cells, which depend upon the constitutive tyrosine kinase activity of the BCR-ABL1 fusion protein for growth¹⁸. However, the high specificity which allows molecularly targeted therapies to selectively attack cancerous cells, also allows heterogeneous cancer cells to evade these targeted therapies. This introduces the second technical problem.

Heterogeneous tumor cells can evade specificity-based anti-tumor therapies when the specific characteristic targeted by the therapy is altered in different cancer cells within the tumor. For example, resistance to imatinib can be mediated through mutations in BCR-ABL1 which alter the conformation of the imatinib binding site, or by amplification of the BCR-ABL1 gene¹⁹⁻²². It has been suggested that persistent administration of imatinib may select for the growth of resistant clones in some patients¹⁹. To address tumor heterogeneity, a new characteristic which is shared by the majority of cells which escaped the first round of treatment must be identified and a new approach must be developed and tested to target the remainder of the tumor. However, the identification of new targets, the development of new molecular agents, and validation of these agents, is a lengthy and expensive process.

In summary, a successful approach for cancer treatment must be highly specific to effectively destroy the cancer at the least cost to the health of the individual, but must also be quickly manipulated to address tumor heterogeneity. The contents of this patent describe one such approach.

### Solution to Technical Problems

Chromosomal rearrangements are a defining characteristic of cancer cells and occur only in cancerous cells, or in those cells en route to entering a cancerous state. The reshuffling of the genetic material in chromosomal rearrangements such as translocations, inversions, insertions, deletions, and duplications physically relocates sequences of DNA and brings together regions of DNA which were previously separate. The junction sites of these chromosomal rearrangements consist of a region of DNA from a first mammalian chromosome into which a chromosomal region from a second mammalian chromosome has been inserted, thus forming a junction between regions of DNA from different chromosomes. These rearrangements can also occur intra-chromosomally, in which a region from a first mammalian chromosome is inserted into a first mammalian chromosome. The junction site of chromosomal rearrangements consists of a new sequence of DNA. In this way, chromosomal rearrangements create sequence specific motifs. Since chromosomal rearrangements are only found in cancerous cells, the sequence specific motifs created by chromosomal rearrangements are cancer specific. These cancer specific sequence motifs (CSSMs) can be used to specifically target cancerous cells for genetic manipulation.

The specific genome editing of cancerous cells through CSSMs requires the use of targetable, sequence specific nucleases, such as zinc finger nucleases (ZFNs), transcription activator-like effector nucleases (TALENs), clustered regularly interspaced short palindromic repeats (CRISPR/Cas9), meganucleases, or chemical nucleases delivered in complement with a cancer specific vector, which serves as the donor DNA. The cancer specific vector is comprised of a sequence of interest as well as first and second targeting arms homologous to the chromosomal rearrangement site. The engineered nucleases bind to the CSSM and induce a double-stranded break (DSB). The naturally occurring homology-directed repair (HDR) pathway in the cell will then utilize the homologous first and second targeting arms on the cancer specific vector to repair the DSB. When a sequence of interest, such as a gene, is positioned between the first and second targeting arms, the HDR process will lead to integration of the sequence of interest into, or near, the CSSM site. This yields cancer specific genome editing. The shortening of the first and second targeting arms to a length of approximately 5-25 bp can lead to the microhomology-mediated end-joining (MMEJ) pathway, which can also be used to insert a sequence of interest into, or near, the CSSM site. Alternatively, engineered nucleases can be delivered without the cancer specific vector, leading to the non-homologous end joining (NHEJ) pathway. Repair of the DSB through NHEJ often yields frameshifting indel mutations that can lead to knockout of fusion gene function, with debilitating consequences for the cancerous cell.

Accordingly, the present invention features nucleic acid constructs, comprised of a sequence of interest, as well as first and second targeting sequences that direct insertion of the sequence of interest into or near the chromosomal rearrangement site (the CSSM); and, optionally, a selectable marker. These sequences can be readily selected and inserted into the nucleic acid constructs using methods well known in the art^{23,24}. The sequence of interest may be operably linked to a regulatory sequence comprising a promoter, when applicable.

Thus, in one aspect, the invention provides cancer specific vectors comprising: A cancer specificity element, comprising a sequence of interest as well as first and second targeting sequences, wherein each of the first and second targeting sequences are homologous to at least 10 bp (e.g., 25, 50, 75, 100, 200, 500, 750, 1000) of sequence located within a genomic distance of no more than 1 MB (e.g., within 1 MB, 0.5 MB, 0.1 MB, 0.05 MB, 10000 KB, 5000 KB, 1000 KB, 500 KB, 100 KB, 50 KB, 10 KB, 5 KB, or 1 KB) from the chromosomal rearrangement site; and a promoter operably linked to the sequence of interest within the cancer specificity element.

Homologous is defined herein as any percent of sequence identity sufficient to facilitate homologous recombination. Such amounts include, without limitation, 50%, 60%, 70%, 80%, 85%, 90%, ₉₅%, 100%, and any values in-between or lower.

Accordingly, the choice of the appropriate percent of sequence identity is within the level of skill in the art. The percent of sequence identity can be determined using the BLASTN program, with default parameters, to obtain homology between nucleotide sequences^{25,26,27}.

The sequence of interest is a sequence of DNA of variable length that may be at least or about 1, 10, 100, 200, 500, 750, 1000, 2000, 5000, 7500, 10000, 15000, 20000 bp (or any integer value in between). Possible sequences of interest include, but are not limited to, cytotoxic genes such as diphtheria toxin, Pseudomonas exotoxin A, streptolysin O, melittin, or gef which can be specifically integrated into the cancer cells and subsequently expressed, killing the cancerous cells as well as cells in the surrounding tumor tissue through the bystander effect²⁸⁻³². Apoptosis inducing genes such as Caspase-3, Rev-Caspase-3, Rev-Caspase-6, iCaspase-9, and Caspase-14 can also be integrated to kill the cancer cell³³⁻³⁶ . The HSV-TK gene is another potential sequence of interest and can be combined with ganciclovir, or other permissible agents such as penciclovir, to selectively kill cancer cells³⁷. Immune stimulatory genes such as GM CSF, CD-40L, CD-28, or proinflammatory cytokines such as IL-12, can be expressed in cancerous cells, with the intent of causing the cancerous cells to promote an immune-stimulatory program ultimately leading to immune system mediated cancer cell death and, in some instances, the abscopal effect³⁸⁻⁴³. A similar approach involving the use of small RNA molecules such as siRNAs or miRNAs intended to silence genes which promote the tumor microenvironment could consist of RNA molecules targeting mRNA from PD-L1, PD-L2, CD39, CD73, COX2, CSF1, A2A and A2B receptors, HIF1α and SP1⁴⁴⁻⁶⁶. The targeting of these genes is intended to abrogate the tumor microenvironment and/or deregulate the oncogenic effect of the tumor microenvironment on tumor cells. Other potential genes of interest for insertion include functional p53, APC, or dominant-negative β-catenin sequences.

Additionally, more than one gene can be introduced into the cancerous cells allowing for greater control over the cancerous cell. These aforementioned examples do not discuss all possible implementations and are not intended to limit the scope of the present disclosure.

The targeting of CSSMs with engineered nucleases and a cancer specific vector can be used to specifically target cancer cells for genetic manipulation. In the event of tumor heterogeneity, a new, patient specific treatment option can be quickly and cheaply developed.

### DESCRIPTION OF DRAWINGS

The drawings described herein are for illustrative purposes only of selected embodiments and not all possible implementations, and are not intended to limit the scope of the present disclosure.

TABLE 1. Nucleotide sequences from normally occurring BCR, the BCR/ABL1 fusion gene, and the normally occurring ABL1. In this example, the nucleotide sequence of the BCR/ABL1 fusion gene is the CSSM. One TALEN can be designed to target the BCR gene region of the CSSM and one TALEN can be designed to target the ABL1 gene region of the CSSM. Catalytic active of the Fok1 nuclease domains is dependent upon dimerization of the two domains, which occurs solely in cells with the BCR/ABL1 fusion gene.

TABLE 2. A list of first and second chromosomal rearrangement sequences which can be targeted by the first and second homologous targeting arms of the cancer specific vector to mediate insertion of the sequence of interest into the cancer cell. Examples of cancer types in which these chromosomal rearrangement sequences are commonly found are given in the right most column.

TABLE 3. A list of sequences of interest which may be inserted into the cancer specific vector, ultimately intended to be integrated into cancerous cells. The intended effect of the expression of said sequences of interest is given in the right most column.

FIG. 1. Nucleotide sequences for the BCR/ABL1 fusion gene CSSM with potential TALEN targeting nucleotides underlined. In this example, the left TALEN recognizes a region from the BCR nucleotide sequence, while the right TALEN recognizes a region from the ABL1 nucleotide sequence. The Fok1 nuclease domains of the TALEN pair dimerize, creating a catalytically active nuclease domain, which induces a double-strand break (DSB) at the junction site of the BCR-ABL1 fusion sequence.

FIG. 2. The Fok1 nuclease domains of the TALEN pair dimerize, creating a catalytically active nuclease domain, which induces a double-strand break (DSB) at the junction site of the BCR-ABL1 fusion sequence. In the absence of donor DNA, the cell undergoes non-homologous end joining (NHEJ) to repair the DSB. NHEJ is error prone and generates indel mutations at the DSB site.

FIG. 3. The Fok1 nuclease domains of the TALEN pair dimerize, creating a catalytically active nuclease domain, which induces a double-strand break (DSB) at the junction site of the BCR-ABL1 fusion sequence. In the presence of donor DNA with short homologous targeting arms (5-25 bp) the microhomology-mediated end joining (MMEJ) repair process can occur, leading to integration of the sequence of interest (SOI) into the DSB site. This process is error prone and deletions can occur in the sequences flanking the SOI.

FIG. 4. The Fok1 nuclease domains of the TALEN pair dimerize, creating a catalytically active nuclease domain, which induces a double-strand break (DSB) at the junction site of the BCR-ABL1 fusion sequence. In the presence of donor DNA with short homologous targeting arms (5-25 bp) positioned directly adjacent in the reverse orientation to their position in the genome causes insertion of the entire donor DNA vector into or near the DSB site through MMEJ.

FIG. 5. The Fok1 nuclease domains of the TALEN pair dimerize, creating a catalytically active nuclease domain, which induces a double-strand break (DSB) at the junction site of the BCR-ABL1 fusion sequence. In the presence of donor DNA with homologous targeting arms, typically 750 bp in length, the cell undergoes homology directed repair (HDR), leading to integration of the sequence of interest (SOI) into or near the DSB site.

FIG. 6. Two sequences from the genome form a translocation, generating a CSSM. The region from one of the two sequences in the fusion sequence is referred to as the ‘first cancer specific rearrangement sequence’ while the region from the remaining sequence is referred to as the ‘second cancer specific rearrangement sequence.’ Cancer specific rearrangement sequences extend no more than 1 MB from the site of the translocation. Together the cancer specific rearrangement sequences form the CSSM. First and second targeting arms of the cancer specific vector share sufficient homology, or sufficient sequence identity, to the first and second cancer specific rearrangement sequences, respectively, to undergo HDR, or in some instances, MMEJ, with the CSSM.

### DETAILED DESCRIPTION

Example embodiments will now be described more fully with reference to the accompanying drawings. Example embodiments do not limit the scope of the invention described in the claims.

Genome editing methods for gene therapy applications have been established for use in an extensive variety of organisms⁶³⁻⁶⁶. These methods involve the use of sequence specific endonucleases to induce a DSB at a target site. The nucleases can be naturally-occurring, such as meganucleases, or engineered to cleave the sequence of interest^{61,62}. Engineered nucleases include zinc finger nucleases (ZFNs), transcription activator-like effector nucleases (TALENs), and the clustered regularly interspaced short palindromic repeats (CRISPR/Cas9) system^{59,63-67}. Genome editing occurs through three primary pathways, non-homologous end joining (NHEJ), homology directed repair (HDR), and microhomology-mediated end-joining (MMEJ), all three of which are initiated by the formation of a double-stranded break (DSB) at a selected site by engineered nucleases. NHEJ is a naturally occurring process which leads to the imperfect repair of the DSB often causing frameshifting indel mutations and is used to knockout gene function. However, NHEJ can be used for imprecise insertion of a DNA vector given that the target site cleaved by the engineered nucleases is present in the donor DNA vector⁶⁸. NHEJ is active during all stages of the cell cycle. HDR is another naturally occurring process within cells in which a DSB is repaired by homologous strand invasion leading to a perfectly repaired sequence. This mechanism can be used to integrate sequences of interest into the genome of the cell. By supplying a donor DNA vector, which may be a circular vector or a linear DNA molecule, containing a sequence of interest to be inserted, homologous targeting arms, and optionally, a reporter gene and, one or more selectable markers, will induce the cell to use the supplied donor DNA to repair the DSB. This leads to homologous strand invasion of the supplied donor DNA into the DSB repair site, guided by the homologous targeting arms, resulting in the use of the donor DNA as the repair template for the DSB. The repair process leads to incorporation of the sequence of interest in the donor DNA into the genome. HDR is active during late S-phase and G2 of the cell cycle. The last repair process, MMEJ, is initiated with the induction of a DSB and relies on short homologous sequences, approximately 5-25 bp in length, to error-prone repair of the DSB⁶⁸. When the short homologous targeting sequences are present in the donor DNA vector, and flank the sequence of interest, MMEJ will lead to integration of the sequence of interest into or near the DSB site. When short homologous targeting sequences are directly adjacent in the donor DNA and in the reverse orientation, as to their orientation in the genome, then the entire donor DNA vector will be inserted into the genome⁶⁸. MMEJ is active during the G₁ and early S phases of the cell cycle.

Chromosomal rearrangements are a defining characteristic of cancer cells and occur primarily in cancerous cells, or in those cells en route to entering a cancerous state. The reshuffling of the genetic material in chromosomal rearrangements such as translocations, inversions, insertions, deletions, and duplications physically relocates sequences of DNA and brings regions of DNA together which were previously separate. The junction site of these chromosomal rearrangements consists of a new sequence of DNA, generated by the two newly adjoining regions. In this way, chromosomal rearrangements create sequence specific motifs. Since chromosomal rearrangements are only found in diseased cells, the sequence specific motifs created by chromosomal rearrangements are disease specific. The cancer specific sequence motifs (CSSMs) can be used to specifically target cancerous cells for genome editing.

The selection of specific chromosomal rearrangements to target for genome editing is guided by the reoccurrence frequency of the chromosomal rearrangement. For example, in chronic myelogenous leukemia (CML), the t(9;22)(q34;q11) translocation, which forms the Philadelphia Chromosome, and the BCR-ABL oncogene, is present in approximately 95% of CML cells⁶⁹. The high reoccurrence frequency of this translocation designates it as a reliable, broad spectrum target for genetic manipulation in the majority of CML cells. Other attractive chromosomal rearrangements for targeting include, but are not limited to, the EWS-FLI1 gene fusion found in 90% of Ewing's Sarcoma cells⁷⁰, the PAX3-FKHR gene fusion found in 55% of alveolar rhabdomyosarcoma⁷¹, or the FLT3 internal tandem duplication (ITD), though found only in a quarter of patients⁷², the FLT3 ITD induces a particularly aggressive hematologic malignancy prone to rapid relapse⁷³. The selection of reoccurring chromosomal rearrangements for genetic manipulation not only provides a consistent target for cancer specific genome editing, but also allows for the disruption of a genetic element necessary for the vitality of the cancerous cell. Reciprocal translocations, such as the translocation found on der (9) in CML are very attractive.

The BCR-ABL fusion gene is one example of a cancer specific sequence motif (CSSM) created by a translocation. The fusion gene is created when the ABL1 gene on chromosome 9q34 is repositioned into the BCL gene on chromosome 22q11⁷⁴. At the nucleotide level, the sequence for the BCL gene is interrupted by the ABL1 gene sequence. In the K562 chronic myelogenous leukemia cell line model, the BCR-ABL translocation on the Philadelphia Chromosome at nucleotide resolution appears as shown in Table 1.

### Table 1.

TABLE 1

| Gene | Sequence |
|---|---|
| BCR | 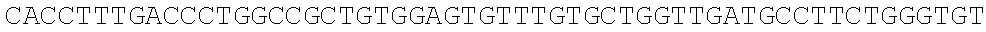 |
| ABL1 | 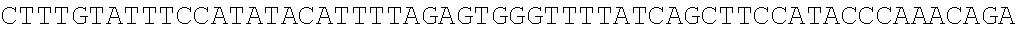 |
| BCR/ABL1 (CSSM) | 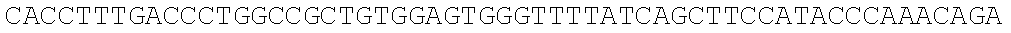 |

The sequence of the translocation, in total, is distinct from either of the two original chromosomal sequences, allowing for the individuation between the BCR/ABL1 fusion gene and both the BCR and ABL1 genes. This permits the individuation between cancerous and non-cancerous cells. Programmable target nucleases including ZFNs, TALENs, CRISPR/Cas9, or chemical nucleases can be used to bind to and induce a DSB into the CSSM sequence.

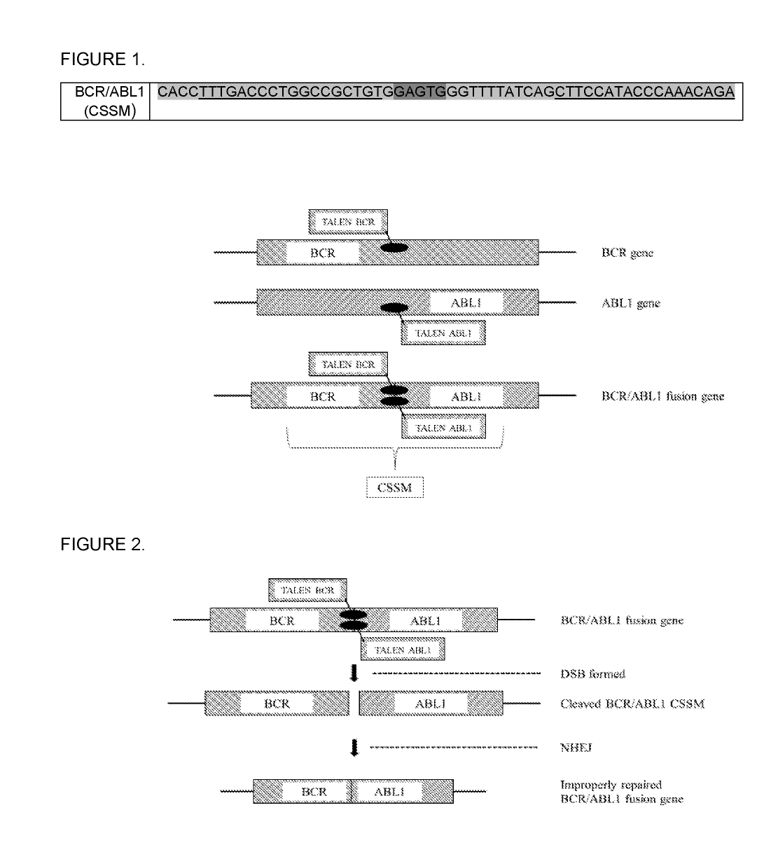

One possible approach involves the use of TALENs to target the BCR/ABL1 CSSM and begins with the design of a pair of TALENs, with one TALEN recognizing the BCR gene while the other TALEN targets the ABL1 gene, as demonstrated in FIG. 1. The TALEN pair recognizes the BCR-ABL1 CSSM and induces a double-strand break (DSB) near the junction site of the two sequences. For the DSB to occur, the TALEN pair must be in close proximity to allow the Fok1 nuclease domains to dimerize, forming the catalytically active nuclease domain. If the Fok1 domains are unable to dimerize, the domains remain inactive and no DSB is induced. Thus, in normal cells wherein the BCR and ABL1 genes are located on different chromosomes, the TALEN pair is inactive since no dimerization between the Fok1 nuclease domains is possible, also shown in FIG. 1. However, when the BCR and ABL1 genes are translocated and become fused together, the TALEN pair becomes catalytically active, cleaving the fusion gene. This technique is not limited to fused genes, but can be used to target any abnormally fused sequences in the genome.

Abnormally fused sequences can form as a result of all types of chromosomal rearrangements including, but not limited to, translocations, inversions, insertions, deletions, and duplications.

In some embodiments, targeting of the CSSM by engineered nucleases is intended to induce non-homologous end joining (NHEJ) causing mutations or insertion of the entire donor DNA vector, as shown in FIG. 2.

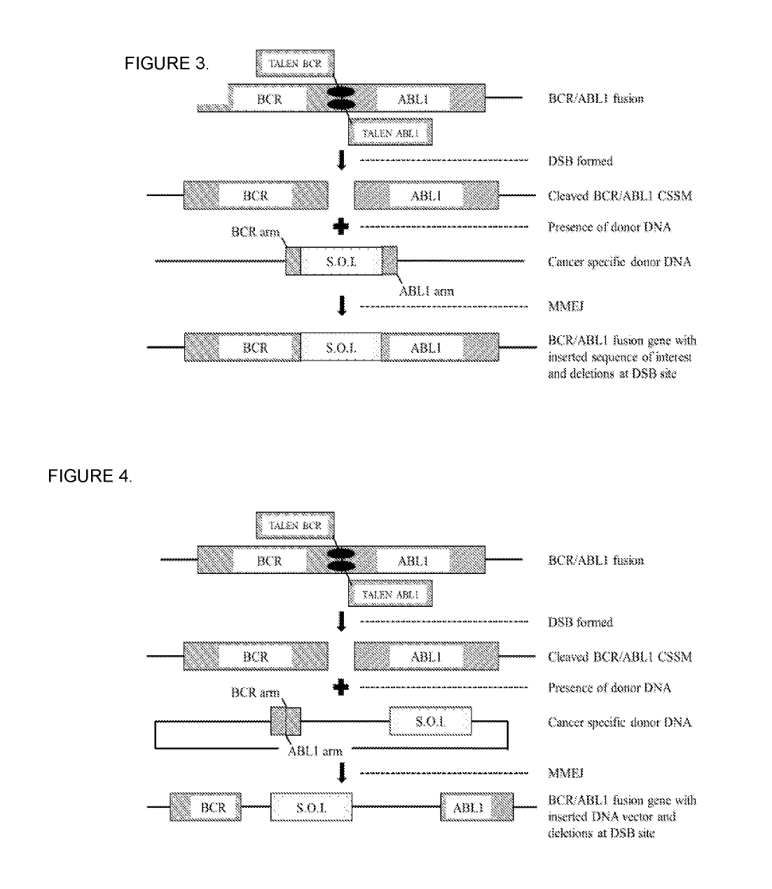

In some embodiments, targeting of the CSSM by engineered nucleases is intended to induce microhomology-mediated end-joining (MMEJ) causing insertion of a fragment of the donor DNA vector or the entirety of the donor DNA vector into or near the site of the DSB, as shown in FIGS. 3 and 4.

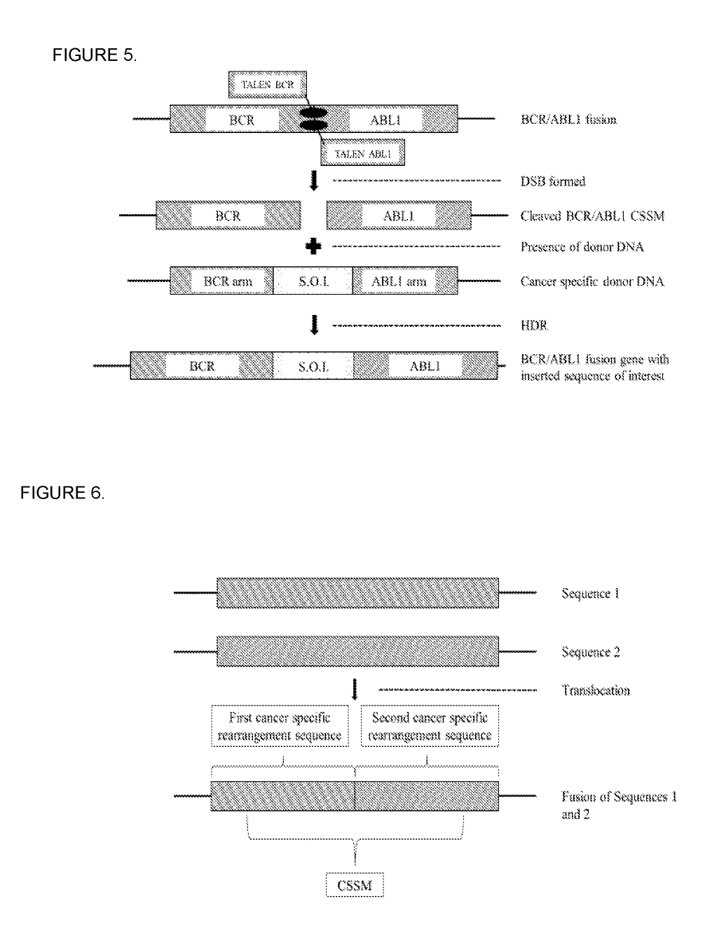

In some embodiments, the DSB is meant to induce homology directed repair (HDR) aimed at the integration of a sequence of interest into the CSSM, as shown in FIG. 5. This approach is further discussed here.

HDR is a naturally occurring process within cells in which a DSB is repaired by homologous strand invasion leading to a perfectly repaired sequence and can be used to integrate sequences of interest into the genome of the cell by supplying donor DNA with a sequence of interest flanked by targeting arms homologous to the regions flanking the DSB^{75,76}. The donor DNA includes the sequence of interest flanked by two homologous arms, typically 750 bp in length, which consist of a sequence of nucleotides homologous to the DNA strands on either side of the DSB. Induction of the DSB by the TALEN pair followed by HDR integrates the sequence of interest into the genome, as shown in FIG. 5. The first and second targeting arms are selected such that they flank the CSSM. These arms target the rearrangement site, which are not adjacent to each other in normal cells. The targeting sequences are chosen such that they enable the targeting sequences to integrate the gene of interest by homologous recombination into the chromosomal region flanked by the first and second cancer specific rearrangement sequences. Choosing homologous targeting sequences to direct insertion into a region of interest is done routinely⁷⁸.

Herein, the first and second cancer specific rearrangement sequences refer to the naturally-occurring, nucleotide sequences flanking, and forming, a CSSM. These rearrangement sequences are adjacent due to the translocation and are therefore cancer specific. The use of the terms ‘first’ and ‘second’ denote that the translocation is formed by two sequences, and do not imply sequential order, nor do they imply the chromosome from which the sequence is originally derived. Refer to FIG. 6 for a diagrammatical representation of first and second cancer specific rearrangement sequences.

Following genome editing, integrated sequences of interest can be expressed, when said expression is driven by a regulatory region. The regulatory region can include a promoter, which may be constitutively active, inducible, tissue-specific, or developmental stage-specific.

In the event that the CML cell is not targeted, a small biopsy of tissue can be extracted and sequenced. From the sequencing data, a new chromosomal rearrangement can be identified and a nuclease can be engineered, along with a targeting vector, to target the newly identified chromosomal rearrangement. Alternatively, this genome editing strategy can be used in concert with other therapeutic strategies to effectively ablate the tumor.

In some embodiments, the donor DNA can be supplied simultaneously with the TALEN pair.

This method, and variations thereof, can be used to selectively target CSSMs generated by chromosomal rearrangements. This offers a novel, bona fide approach for genome editing in a cancer specific fashion.

Nucleic Acid Constructs—Cancer Specific Vectors

The cancer specific vector or donor DNA (the terms are used interchangeably herein) is the DNA molecule, which contains the sequence of interest, the homologous targeting sequences (e.g., first and second targeting sequences that direct integration into the CSSM), and a regulatory region comprising a promoter operably linked to the sequence of interest, when applicable. The cancer specific vector can also include a selectable marker, whose expression is driven by the aforementioned regulatory region, or in some instances, by a different regulatory region. The regulatory region can include a promoter, which may be constitutively active, inducible, tissue-specific, or developmental stage-specific. Additional regulatory elements such as enhancers and polyadenylation sequences may also be included.

Cancer Specific Vectors—Cancer Specificity Element

The cancer specificity element is comprised of the sequence of interest, operably linked to a promoter when applicable, and the first and second homologous targeting sequences. It is the cancer specificity element, which serves as the template for HDR, and in some instances of MMEJ, and leads to integration of the sequence of interest and the accompanying regulatory region(s).

Cancer Specific Vectors—Homologous Targeting Sequences

The nucleic acid vectors described herein include homologous arms or targeting sequences (the terms are used interchangeably herein) that direct sequence specific integration of the donor DNA into the chromosomal rearrangement site through HDR or MMEJ. Targeting sequences may vary in size. In certain embodiments, a targeting sequence may be at least or about 5, 10, 25, 50, 100, 200, 300, 400, 500, 600, 700, 800, 900, 1000, 2000, 3000, 4000, 5000, 6000, 7000, 8000, 9000, 10000 bp in length (or an integer value in between). The targeting elements as described here may be variants of naturally occurring genomic sequences. In certain embodiments, a targeting sequence is homologous to the sequence at the chromosomal rearrangement site that occurs naturally within the cancer cell, referred to herein as chromosomal rearrangement sequences. Every translocation is comprised of two chromosomal rearrangement sequences, as shown in FIG. 6. Preferably, the targeting sequence includes a nucleotide sequence at least 75% identical to its corresponding naturally occurring sequence (the chromosomally rearranged genomic sequence). More preferably, the nucleotide sequence is 90%-100%, or any percentage value in between, identical to the reference sequence, although any percent of sequence identity sufficient to induce homologous recombination or microhomology-mediated repair is viable.

Cancer Specific Vectors—Homologous Targeting Sequences—Examples

Homologous targeting sequences can be designed to share sufficient sequence identity with the first and second genes in the fusion, also referred to as first and second chromosomal rearrangement sequences, enabling the cancer cell to be specifically targeted. See Table 2 for a list of potential genes which may be used to target the CSSM of specific cancer types.

TABLE 2

| CSSM | | |
|---|---|---|
| 1^{st} Chr. | 2^{nd} Chr. | |
| Rearrange. Seq. | Rearrange. Seq. | Cancer Type |
| BCR | ABL1 | Chronic Myelogenous Leukemia |
| | | Acute Lymphoblastic Leukemia |
| CBFB | MYH11 | Acute Myeloid Leukemia |
| CRTC1 | MAML2 | Mucoepidermoid Carcinomas |
| DEK | NUP214 | Acute Myeloid Leukemia |
| EML4 | ALK | Non-Small Cell Lung Cancer |
| EWSR1 | ATF1 | Hyalinizing Clear-Cell Carcinoma |
| EWSR1 | ERG | Ewings Sarcoma |
| EWSR1 | ETS | Ewings Sarcoma |
| EWSR1 | FLI1 | Ewings Sarcoma |
| FGFR3 | TACC3 | Head and Neck Squamous Cell |
| | | Carcinoma |
| FLT3 | FLT3 | Acute Myeloid Leukemia |
| FUS | ERG | Acute Myeloid Leukemia |
| FUS | DDIT3 | Myxoid/Round Cell Liposarcoma |
| KIAA1549 | BRAF | Pilocytic Astrocytomas |
| KMT2A | AF4 | ALL, Acute Myeloid Leukemia |
| KMT2A | AF9 | ALL, Acute Myeloid Leukemia |
| KMT2A | ENL | ALL, Acute Myeloid Leukemia |
| MYB | NFIB | Adenoid Cystic Carcinoma |
| | | Dermal Cylindroma |
| NDRG1 | ERG | Prostate |
| NPM1 | ALK | Anaplastic Large Cell Lymphoma |
| | | Acute Myeloid Leukemia |
| PAX3 | FOXO1 | Alveolar Rhabdomyosarcoma |
| PAX7 | FOXO1 | Alveolar Rhabdomyosarcoma |
| PAX8 | PPARG | Thyroid Carcinoma |
| PML | RARA | Acute Promyelocytic Leukemia |
| RUNX1 | RUNX1T1 | Acute Myeloid Leukemia |
| TCF3 | PBX1 | Acute Lymphoblastic Leukemia |
| TMPSSR2 | ERG | Prostate |

Cancer Specific Vectors—Sequences of Interest

As mentioned, the sequence of interest can be any nucleic acid sequence, which when transcribed, and in some instances, translated, in the cell alters some aspect of cellular behavior preferably resulting in the death of the cancer cell, or rendering the cancer cell less threatening to the health of the patient. The gene or sequence of interest can be, but is not limited to, nucleic acid sequence coding for a marker protein, a cytotoxin, a suicide gene, a tumor suppressor gene, an shRNA, an miRNA, or a ribozyme. See Table 3 for examples of desired genes.

TABLE 3

| Sequence Name | Sequence Type | Intended Function |
|---|---|---|
| Caspase-3 | Gene/cDNA | Apoptosis |
| Caspase-14 | Gene/cDNA | Apoptosis |
| iCaspase-9 | Gene/cDNA | Apoptosis |
| Rev-Caspase-3 | Gene/cDNA | Apoptosis |
| Rev-Caspase-6 | Gene/cDNA | Apoptosis |
| TP53 | Gene/cDNA | Apoptosis |
| Diphtheria toxin | Gene/cDNA | Cytotoxicity |
| Gef | Gene/cDNA | Cytotoxicity |
| Melittin | Gene/cDNA | Cytotoxicity |
| Pseudomonas exotoxin A | Gene/cDNA | Cytotoxicity |
| Streptolysin O | Gene/cDNA | Cytotoxicity |
| CD-28 | Gene/cDNA | Immune Stimulation |
| CD-40L | Gene/cDNA | Immune Stimulation |
| GM CSF | Gene/cDNA | Immune Stimulation |
| IL-12 | Gene/cDNA | Immune Stimulation |
| Cytosine Deaminase | Gene/cDNA | Induced Cytotoxicity |
| HSV-TK | Gene/cDNA | Induced Cytotoxicity |
| APC | Gene/cDNA | Regulate Wnt signaling |
| Dominant-negative | Gene/cDNA | Regulate Wnt signaling |
| CTNNB1 | | |
| A_{2A} Receptor | miRNA/shRNA | Abrogate |
| | | Microenvironment |
| A_{2B} Receptor | miRNA/shRNA | Abrogate |
| | | Microenvironment |
| CD39 | miRNA/shRNA | Abrogate |
| | | Microenvironment |
| CD73 | miRNA/shRNA | Abrogate |
| | | Microenvironment |
| COX2 | miRNA/shRNA | Abrogate |
| | | Microenvironment |
| CSF1 | miRNA/shRNA | Abrogate |
| | | Microenvironment |
| HIF1α | miRNA/shRNA | Abrogate |
| | | Microenvironment |
| PD-L1 | miRNA/shRNA | Abrogate |
| | | Microenvironment |
| PD-L2 | miRNA/shRNA | Abrogate |
| | | Microenvironment |
| SP1 | miRNA/shRNA | Abrogate |
| | | Microenvironment |

Cancer Specific Vectors—Optional Selection Markers

Selection markers may be found within the cancer specific targeting vector for the selection of successfully transfected, or transformed, (the terms are used interchangeably) cells. Drug resistance genes to antibiotics, such as neomycin or G418 are popular selection markers in human cells. Generally, the term “marker” refers to a gene or sequence whose presence, or absence, conveys a detectable phenotype. The two most commonly used types of markers are selection markers and screening markers. Selection markers are genes which, when expressed, convey resistance to a specific set of environmental conditions. Typically, only cells with the vector containing the selection marker will be able to proliferate, thereby selecting the successfully transfected cells. Screening markers convey a readily identifiable phenotype, allowing for the differentiation between transfected and non-transfected cells. In a screen, all cells can proliferate, however only transfected cells display the identifiable phenotype conveyed by the vector and can be chosen based thereof.

Double-Strand Break (DSB) Inducing Vectors

The present description includes the use of a DSB-inducing vector, i.e., a nucleic acid construct which includes a sequence that enhances or facilitates HDR or MMEJ by introducing a DSB break at the target site (e.g., encodes a ZFN, TALEN, or CRISPR). These programmable nucleases can recognize and target specific chromosomal sequences to facilitate targeted integration of the sequence of interest into the target site, in this description, the site of the chromosomal rearrangement (CSSM). As is understood in the art, HDR and MMEJ are the processes by which a DSB break is repaired in the genetic material at a specified locus mediated by the use of homologous DNA sequences. Introduction of the ZFN, TALEN, of CRISPR, which mediate the formation of the DSB at the specified site can take several forms. These include introducing the ZFN, TALEN, or CRISPR, as part of a nucleic acid construct, as part of a strand of mRNA, or the protein of the ZFN, TALEN, or CRISPR itself.

The introduction of the vectors, the cancer specific targeting vector and the DSB- inducing vector, can be performed in a variety of ways. Additionally, the active sequences in the cancer specific targeting vector and the DSB-inducing vector can be introduced to the host cell on the same vector or separately (e.g., on separate vectors or separate types of vectors at the same time or sequentially). These methods are now discussed and are well known in the art.

Transformations, or transfections, can be performed through a variety of techniques depending upon the particular requirements of each cell type or organism. These techniques have been extensively tested and are readily adaptable to different cells and organisms.

Liposomal formulations involve the addition of a liposomal reagent in addition to the cancer specific vector and the DSB-inducing vector. Liposomes are vesicular structures formed by a phospholipid membrane with an inner aqueous medium. Vesicular structures self-assemble in aqueous solution due to the hydrophilic and hydrophobic tendencies of the phospholipid molecules. The formation of vesicles by phospholipid molecules entraps water and dissolved solutes, such as DNA, between the lipid bilayers⁷⁹. Liposomal formulations can hold different charges allowing them to interact with DNA, RNA, as well as other substances and can be formulated to target specific cell receptors on the cell membrane⁸⁰⁻⁸². One such example, cationic lipids, form complexes with nucleic acids and fuse with the membrane of the cell^{83,84}. This process increases the efficiency of transformation or transfection. Recent advances include the application of microfluidic mixing used to formulate liposomal particles, which has led to increased encapsulation and transfection efficiency⁸⁵. Lipids and liposomes suitable for use in delivering the present vectors can be obtained from commercial sources.

Direct microinjection of the nucleic acid vectors into various cells is also contemplated, and has been used effectively in genome editing applications across a variety of species. The nucleic acid is simply injected into the cytoplasm or nucleus of the cell of interest⁸⁶.

Viral vectors may also be employed in the present invention to mediate delivery of the nucleic acid vectors in vitro, ex vivo, or in vivo. Reovirus, Newcastle Disease virus, Mumps virus, Moloney leukemia virus, measles, vesicular stomatitis virus, Vaccinia virus, adenovirus, adeno-associated virus, rabies virus, pox virus, human foamy virus, lentivirus, and herpes simplex viruses may be attenuated and used. Viral vector genomes have been modified to inhibit replication and normal viral function, enabling them for safe, therapeutic use. Additionally, viral tropism can be used as another method of selection for cancerous cells. Successful viral mediated in vivo genome editing in humans has been demonstrated with an adeno-associated viral (AAV) vector encoding the blood coagulation factor IX (F.IX), introduced to skeletal muscle via an intramuscular injection in haemophilia B patients⁸⁷. Expression of F. IX was detectable at low vector doses⁸⁷. A modified herpes simplex virus (T-VEC) expressing the granulocyte-macrophage colony-stimulating factor (GM-CSF) has been used in a phase III clinical trial to treat patients with unresected stage IIIB-IV metastatic melanoma through intratumoral injection88. T-VEC administration proved to be therapeutically beneficial resulting in significantly increased durable response rate and a longer median overall survival⁸⁸. The viral vectors mediating the delivery of the nucleic acid vectors described herein can be administered intratumorally, intramuscularly, intracerebrally, or intravenously, depending upon the nature of the cancer in the patient.

In one embodiment, the invention features pharmaceutically acceptable compositions that include the nucleic acid vectors described herein. Various combinations of the vectors described herein can be formulated as pharmaceutical compositions.

Also within the scope of this invention are RNAs and proteins encoded by the DSB-inducing vector and compositions that include them (e.g. lyophilized preparations or solutions, including pharmaceutically acceptable solutions or other pharmaceutical formulations).

In another aspect, the invention provides cells produced by a method described herein. The cells can be mammalian cells, such as human cells, monkey cells, rat cells, mouse cells, etc. In some embodiments, the cell is a stem cell. In some embodiments, the stem cell is an induced pluripotent stem cell (iPSC), a neural stem cell, or a hematopoietic stem cell.

A cancer specific vector comprising sequences shown in SEQ. ID. NOs.: 1, 2, 3, 4, 5, 6, 7, 8, 9, 10, 11, 12, 13, 14, 15.

Method of Treatment

The methods of the invention can be used to treat patients who have a cancerous tumor, composed of cells with an identified chromosomal rearrangement. Any of the methods can include the step of identifying a patient in need of treatment; any of the patients can be human; and any of the methods can include the step of identifying the chromosomal rearrangement present in the cells of the tumor, and any of the methods can be performed by administering the present compositions to the patient. For example, the invention features methods of treating a cancer caused by cancerous cells with a chromosomal rearrangement by identifying a patient in need of treatment, identifying the chromosomal rearrangement in the cancer cells; and administering to the patient a nucleic acid construct, vector, and/or DSB-inducing vector as described herein. The amount of the construct or vector administered will be an amount sufficient to improve a condition associated with the disease. As noted above, treatment can also be performed in vivo by administering present compositions to the patient via pharmaceutically acceptable compositions.

To illustrate a particular application, CSSM targeted genome editing could be used to treat various stages of melanoma. There are two treatment regimes envisioned based on disease state. In a patient with stage I or II melanoma, a biopsy can be taken and sequenced. From the sequencing data, translocations will be identified and their prevalence estimated. A cancer specific vector will be rapidly constructed to target the CSSM of the identified translocation. The cancer specific vector could comprise the herpes simplex thymidine kinase (HSK-TK) gene driven by a constitutively active regulatory element, flanked by first and second homologous targeting arms each of approximately 750 bp in length, each with approximately 95% sequence identity to the first and second chromosomal rearrangement sequences, respectively. A DSB-inducing vector would be rapidly synthesized in parallel, using TALENs targeting the CSSM. TALEN activity would be verified using in vitro translation of the TALENs to cleave a PCR amplified CSSM sequence. The cancer specific vector would be transfected into a melanoma cell line with the addition of ganciclovir to verify potent killing activity. The DSB-inducing vector and the cancer specific vector would be packaged into attenuated vaccinia viruses and delivered through intratumoral injection to the tumor site. The patient would then be treated with ganciclovir or another permissible agent. An identical routine would be performed for a patient with Stage III or IV melanoma, however three cancer specific vectors would be constructed, differing by their gene of interest. The first would contain melittin, the second GM-CSF, and the third would contain an shRNA targeting CD39 mRNA transcripts. The intent of this combination is to impair the tumor microenvironment through CD39 silencing, recruit the immune system with GM-CSF to target local and distant cancer sites, and to kill the local tumor as well as promoting inflammation using melittin. Viruses would be injected intratumorally, though can be injected intravenously as a last resort.

### Advantageous Effects of Invention

The invention contains several advantages over existing cancer treatment strategies. The foremost advantage is the broad-spectrum applicability of the invention, which allows for the specific targeting of any type of chromosome rearrangement. Existing strategies such as small molecule inhibitor based therapies, such as imatinib, rely upon the activity of a protein product of a fusion gene for targeting. However, not all rearrangements involve the formation and expression of a fusion gene, limiting the applicability of small molecule inhibitors. Since all chromosomal rearrangements generate a CSSM, the proposed method can theoretically target all chromosomal rearrangements and is not dependent upon the formation and expression of a fusion gene.

A second advantage is the versatility of the invention. Any sequence of interest can be inserted between the targeting sequences, allowing for a variety of options for manipulating the cancer cell. Although killing the cancer cell is an advantageous use of this invention, the specific method of killing, via immune system activation, toxin production, abrogation of the tumor microenvironment, or the reintroduction of apoptotic or tumor suppressor genes can be chosen. Additionally, the cancer cell can be genetically altered in some other way, which does not induce cancer cell death, such as being genetically reprogrammed to another cell type or decreasing the proliferative or metastatic potential of the cancer cell, which is facilitated in some way by the addition, and in some instances subsequent expression, of any desired nucleotide sequence of interest, including noncoding, coding, RNA, and DNA sequences into the cancerous cell.

A third advantage of the invention is the ability to quickly develop a patient specific therapy. The time required to construct programmable target nucleases to target a new CSSM, a new donor DNA (cancer specific vector), and to verify their activity, is approximately three months. This is significantly less than the amount of time required to test a small molecule or design a new monoclonal antibody for immunotherapy. Additionally, the cost required to produce a new target nuclease and donor DNA (cancer specific vector) is significantly less than costs associated with the production of new antibodies or small molecule inhibitors.

Unless otherwise defined, all technical and scientific terms used herein have the same meaning as commonly understood by one of ordinary skill in the art to which this invention belongs. Methods and materials described herein for use in the present invention; other suitable methods and materials known in the art can also be used. The materials, methods, and examples presented herein are illustrative only and not intended to be limiting. All publications, patent applications, patents, sequences, database entries, and other references mentioned herein are incorporated by reference in their entirety. In case of conflict, the present specification, including definitions, will control.

### REFERENCES

1. International Agency for Research on Cancer. World cancer report, 2014. Lyon, France: World Health Organization, International Agency for Research on Cancer; 2014. http.//publications.iarc.fr/Non-Series-Publications/World-Cancer-Reports/World-Cancer-Report-2014
2. Howlader N, Noone A M, Krapcho M, Miller D, Bishop K, Altekruse S F, Kosary C L, Yu M, Ruhl J, Tatalovich Z, Mariotto A, Lewis D R, Chen H S, Feuer E J, Cronin K A (eds). SEER Cancer Statistics Review, 1975-2013, National Cancer Institute. Bethesda, Md., http://seer.cancer.gov/csr/1975_2013/, based on November 2015 SEER data submission, posted to the SEER web site, April 2016.
3. Mariotto A B, Yabroff K R, Shao Y, Feuer E J, Brown M L. Projections of the Cost of Cancer Care in the U.S.: 2010-2020. J Natl Cancer Inst. 2011 January
4. Russell, Stephen J, Kah-Whye Peng, and John C Bell. “ONCOLYTIC VIROTHERAPY.” Nature biotechnology 30.7 (2012): 658-670. PMC. Web. 28 Dec. 2016.
5. Adams J L, Smothers J, Srinivasan R, Hoos A. Big opportunities for small molecules in immuno-oncology. Nat Rev Drug Discov. 2015;14:603-622
6. Khalil, D N, Smith, E L, Brentjens, R J and Wolchok, J D (2016). The future of cancer treatment: immunomodulation, CARs and combination immunotherapy. Nat Rev Clin Oncol 13: 273-290.
7. Knapper S, Burnett A K, Littlewood T, Kell W J, Agrawal S, Chopra R, Clark R, Levis M J, Small D. A phase 2 trial of the FLT3 inhibitor lestaurtinib (CEP701) as first-line treatment for older patients with acute myeloid leukemia not considered fit for intensive chemotherapy. Blood. 2006;108:3262-70.
8. Burrell, R. A., McGranahan, N., Bartek, J., and Swanton, C. (2013a). The causes and consequences of genetic heterogeneity in cancer evolution. Nature 501, 338-345.
9. McGranahan N, Swanton C. Biological and therapeutic impact of intratumor heterogeneity in cancer evolution. Cancer Cell 2015;27:15-26.
10. Bozic, Ivana et al. “Evolutionary Dynamics of Cancer in Response to Targeted Combination Therapy.” Ed. Carl T Bergstrom. eLife 2 (2013): e00747.
11. Genovese, Pietro et al. “Targeted Genome Editing in Human Repopulating Hematopoietic Stem Cells.” Nature 510.7504 (2014): 235-240
12. Hoban, Megan D. et al. “Correction of the Sickle Cell Disease Mutation in Human Hematopoietic Stem/progenitor Cells.” Blood 125.17 (2015): 2597-2604
13. Mendell J R, Rodino-Klapac L R. CRISPR/Cas9 treatment for Duchenne muscular dystrophy. Cell Res. 2016;26(5):513-514.
14. Schwank G, Koo B K, Sasselli V, et al. Functional repair of CFTR by CRISPR/Cas9 in intestinal stem cell organoids of cystic fibrosis patients. Cell Stem Cell 2013;13:653-8. 10.1016/j.stem.2013.11.002
15. Tebas, Pablo et al. “Gene Editing of CCR5 in Autologous CD4 T Cells of Persons Infected with HIV.” The New England journal of medicine 370.10 (2014): 901-910.
16. Cox D B, Platt R J, Zhang F. Therapeutic genome editing: prospects and challenges. Nat Med. 2015;21:121-131.
17. Druker B J, Talpaz M, Resta D J. Efficacy and safety of a specific inhibitor of the BCR-ABL tyrosine kinase in chronic myeloid leukemia. N Engl J Med. 2001;344:1031
18. Effects of a selective inhibitor of the Abl tyrosine kinase on the growth of Bcr-Abl positive cells. Druker B J, Tamura S, Buchdunger E, Ohno S, Segal G M, Fanning S, Zimmermann J, Lydon N B. Nat Med. 1996;2:561-566
19. Gorre ME, Mohammed M, Ellwood K, Hsu N, Paquette R, Rao P N, et al. Clinical resistance to STI-571 cancer therapy caused by BCR-ABL gene mutation or amplification. Science. 2001;293:876-880.
20. Weisberg E, Griffin J D. Mechanism of resistance to the ABL tyrosine kinase inhibitor STI571 in BCR/ABL-transformed hematopoietic cell lines. Blood. 2000;95(11):3498-505
21. le Coutre P, Tassi E, Varella-Garcia M, Barni R, Mologni L, Cabrita G, et al. Induction of resistance to the Abelson inhibitor STI571 in human leukemic cells through gene amplification. Blood. 2000;95(5):1758-66
22. Mahon F X, Deininger MW, Schultheis B, Chabrol J, Reiffers J, Goldman J M, et al. Selection and characterization of BCR-ABL positive cell lines with differential sensitivity to the tyrosine kinase inhibitor STI571: diverse mechanisms of resistance. Blood. 2000;96(3):1070-9.
23. Wu S, Ying G X, Wu Q, Capecchi M R. A protocol for constructing gene targeting vectors: generating knockout mice for the cadherin family and beyond. Nat. Protoc. 2008;3:1056-1076
24. Adachi N, Kurosawa A, Koyama H. Highly proficient gene targeting by homologous recombination in the human pre-B cell line Nalm-6. Methods Mol Biol. 2008;435:17-29. doi: 10.1007/978-1-59745-232-8_2.
25. Karlin, S, and S F Altschul. “Methods for Assessing the Statistical Significance of Molecular Sequence Features by Using General Scoring Schemes.” Proceedings of the National Academy of Sciences of the United States of America 87.6 (1990): 2264-2268. Print.
26. Altschul, S. F., Gish, W., Miller, W., Myers, E. W. & Lipman, D. J. (1990) “Basic local alignment search tool.” J. Mol. Biol. 215:403-410.
27. Boratyn G M, Camacho C, Cooper P S, Coulouris G, Fong A, Ma N, Madden T L, Matten W T, McGinnis S D, Merezhuk Y, Raytselis Y, Sayers E W, Tao T, Ye J, & Zaretskaya I. (2013) “BLAST: a more efficient report with usability improvements.” Nucleic Acids Res. 41:W29-W33.
28. Ayesh B, Matouk I, Ohana P, Sughayer M A, Birman T, Ayesh S, Schneider T, de Groot N, Hochberg A. Inhibition of tumor growth by DT-A expressed under the control of IGF2 P3 and P4 promoter sequences. Mol Ther. 2003;7:535-541.
29. Seth P, Brinkmann U, Schwartz G N, et al. Adenovirus-mediated gene transfer to human breast tumor cells: an approach for cancer gene therapy and bone marrow purging. Cancer Research. 1996; 56(6):1346-1351.
30. Yang W S, Park S O, Yoon A R, Yoo J Y, Kim M K, Yun C O, Kim C W. Suicide cancer gene therapy using pore-forming toxin, streptolysin O. Mol Cancer Ther. 2006;5:1610-1619. doi: 10.1158/1535-7163.MCT-05-0515.
31. Ling C. Q., Li B., Zhang C., Zhu D. Z., Huang X. Q., Gu W., Li S. X. Inhibitory effect of recombinant adenovirus carrying melittin gene on hepatocellular carcinoma. Ann. Oncol. 2005;16:109-115. doi: 10.1093/annonc/mdi019.
32. Prados J, Melguizo C, Ortiz R, Boulaiz H, Carrillo E, Segura A, et al. Regression of established subcutaneous B16-F10 murine melanoma tumors after gef gene therapy associated with the mitochondrial apoptotic pathway. Exp Dermatol. 2010;19:363-71. doi: 10.1111/j.1600-0625.2009.00914.x
33.Yamabe K, Shimizu S, Ito T, Yoshioka Y, Nomura M, Narita M, Saito I, Kanegae Y, Matsuda H. Cancer gene therapy using a pro-apoptotic gene, caspase-3. Gene Ther. 1999;6:1952-1959. doi: 10.1038/sj.gt.3301041.
34. Srinivasula S. M. et al. . Generation of constitutively active recombinant caspases-3 and -6 by rearrangement of their subunits. J. Biol. Chem. 273, 10107-10111 (1998)
35. Song, Wenying et al. “Cancer Gene Therapy with iCaspase-9 Transcriptionally Targeted to Tumor Endothelial Cells.” Cancer gene therapy 15.10 (2008): 667-675. PMC. Web. 21 Jan. 2017.
36. Wu, M et al. “Exogenous Expression of Caspase-14 Induces Tumor Suppression in Human Salivary Cancer Cells by Inhibiting Tumor Vascularization.” Anticancer research 29.10 (2009): 3811-3818
37. Shaw M M, Gurr W K, Watts P A, Littler E, Field H J. Ganciclovir and penciclovir, but not acyclovir, induce apoptosis in herpes simplex virus thymidine kinase-transformed baby hamster kidney cells. Antivir Chem Chemother. 2001;12(3):175-186.
38. Quezada, S. A. CTLA4 blockade and GM-CSF combination immunotherapy alters the intratumor balance of effector and regulatory T cells. J. Clin. Invest. 116, 1935-1945 (2006).
39. Brentjens, R. J. et al. Genetically targeted T cells eradicate systemic acute lymphoblastic leukemia xenografts. Clin. Cancer Res. 13, 5426-5435 (2007).
40. Davila, M. L. et al. Efficacy and toxicity management of 19-28z CAR T cell therapy in B cell acute lymphoblastic leukemia. Sci. Transl. Med. 6, 224ra25 (2014).
41. Pegram, H. J. et al. Tumor-targeted T cells modified tosecrete IL-12 eradicate systemic tumors without need for prior conditioning. Blood 119, 4133-4141 (2012).
42. Zhao, Z. et al. Structural design of engineered costimulation determines tumor rejection kinetics and persistence of CAR T cells. Cancer Cell 28, 415-428 (2015).
43. Curran, K. J. et al. Enhancing antitumor efficacy of chimeric antigen receptor T cells through constitutive CD4OL expression. Mol. Ther. 23, 769-778 (2015).
44. Deaglio, S. et al. Adenosine generation catalyzed by CD39 and CD73 expressed on regulatory T cells mediates immune suppression. J. Exp. Med. 204, 1257-1265 (2007).
45. Borsellino, G. et al. Expression of ectonucleotidase CD39 by Foxp3⁺ Treg cells: hydrolysis of extracellular ATP and immune suppression. Blood 110, 1225-1232 (2007).
46. Sun, X. et al. CD39/ENTPD1 expression by CD4⁺ Foxp3⁺regulatory T cells promotes hepatic metastatic tumor growth in mice. Gastroenterology 139, 1030-1040 (2010).
47. Michaud, M. et al. Subversion of the chemotherapy-induced anticancer immune response by the ecto-ATPase CD39. Oncoimmunology 1, 393-395 (2012).
48. Synnestvedt, K. et al. Ecto-5′-nucleotidase (CD73) regulation by hypoxia-inducible factor-1 mediates permeability changes in intestinal epithelia. J. Clin. Invest. 110, 993-1002 (2002).
49. Stagg, J. & Smyth, M. J. Extracellular adenosine triphosphate and adenosine in cancer. Oncogene 29, 5346-5358 (2010).
50. Clayton, A., Al-Taei, S., Webber, J., Mason, M. D. & Tabi, Z. Cancer exosomes express CD39 and CD73, which suppress T cells through adenosine production. J. Immunol. 187, 676-683 (2011).
51. Mandapathil, M. et al. Adenosine and prostaglandin e2 cooperate in the suppression of immune responses mediated by adaptive regulatory T cells. J. Biol. Chem. 285, 27571-27580 (2010).
52. Wang, D. & DuBois, R. N. The role of anti-inflammatory drugs in colorectal cancer. Ann. Rev. Med. 64, 131-144 (2013).
53. Beavis, P. A. et al. Blockade of A2A receptors potently suppresses the metastasis of CD73⁺ tumors. Proc. Natl Acad. Sci. USA 110, 14711-14716 (2013).
54. Cekic, C. et al. Adenosine A 2B receptor blockade slows growth of bladder and breast tumors. J. Immunol. 188, 198-205 (2012).
55. DeNardo, D. G. et al. Leukocyte complexity predicts breast cancer survival and functionally regulates response to chemotherapy. Cancer Discov. 1, 54-67 (2011).
56. Brahmer, J. R. et al. Safety and activity of anti-PD-L1 antibody in patients with advanced cancer. N. Engl. J. Med. 366, 2455-2465 (2012).
57. Silva, George et al. “Meganucleases and Other Tools for Targeted Genome Engineering: Perspectives and Challenges for Gene Therapy.” Current Gene Therapy 11.1 (2011): 11-27.
58. Joung, J. Keith, and Jeffry D. Sander. “TALENs: A Widely Applicable Technology for Targeted Genome Editing.” Nature reviews. Molecular cell biology 14.1 (2013): 49-55.
59. Sander, Jeffry D., and J. Keith Joung. “CRISPR-Cas Systems for Genome Editing, Regulation and Targeting.” Nature biotechnology 32.4 (2014): 347-355.
60. Urnov F D, Rebar E J, Holmes M C, Zhang H S, Gregory P D. Genome editing with engineered zinc finger nucleases. Nat Rev Genet. 2010;11:636-646. doi: 10.1038/nrg2842.
61. Silva G H, Belfort M, Wende W, Pingoud A. From monomeric to homodimeric endonucleases and back: Engineering novel specificity of LAGLIDADG enzymes. J Mol Biol. 2006;361(4):744-754.
62.Arnould, S, Perez, C, Cabaniols, JP, Smith, J, Gouble, A, Grizot, S et al. (2007). Engineered I-Crel derivatives cleaving sequences from the human XPC gene can induce highly efficient gene correction in mammalian cells. J Mol Biol 371: 49-65
63. Foley, Jonathan E. et al. “Rapid Mutation of Endogenous Zebrafish Genes Using Zinc Finger Nucleases Made by Oligomerized Pool ENgineering (OPEN).” Ed. David W. Raible. PLoS ONE 4.2 (2009): e4348.
64. Maeder, Morgan L. et al. “Rapid ‘open-Source’ Engineering of Customized Zinc- Finger Nucleases for Highly Efficient Gene Modification.” Molecular cell 31.2 (2008): 294-301.
65. Cermak, Tomas et al. “Efficient Design and Assembly of Custom TALEN and Other TAL Effector-Based Constructs for DNA Targeting.” Nucleic Acids Research 39.12 (2011): e82
66. Sakuma T. et al. Efficient TALEN construction and evaluation methods for human cell and animal applications. Genes to Cells 18, 315-326 (2013).
67. Sanjana, Neville E. et al. “A Transcription Activator-Like Effector (TALE) Toolbox for Genome Engineering.” Nature protocols 7.1 (2012): 171-192.
68. Sakuma T, Nakade S, Sakane Y, Suzuki KT, Yamamoto T. MMEJ-assisted gene knock-in using TALENs and CRISPR-Cas9 with the PITCh systems. Nat Protoc. 2016;11:118-133.
69. Rowley J. D. (1973). A new consistent chromosomal abnormality in chronic myelogenous leukemia identified by quinacrine fluorescence and Giemsa staining. Nature. 243: 290-293.
70. Delattre O, Zucman J, Plougastel B, Desmaze C, Melot T, Peter M, Kovar H, Joubert I, de Jong P, Rouleau G, Aurias A, Thomas G. Nature. 1992;359:162-5.
71. Sorensen P. H., Lynch, J. C., Qualman, S. J., Tirabosco, R., Lim, J. F., Maurer, H. M., Bridge, J. A., Crist, W. M., Triche, T. J., and Barr, F. G. 2002. PAX3-FKHR and PAX7-FKHR gene fusions are prognostic indicators in alveolar rhabdomyosarcoma: A report from the children's oncology group. J. Clin. Oncol. 20: 2672-2679.
72. Kiyoi H, Towatari M, Yokota S, Hamaguchi M, Ohno R, Saito H, Naoe T. Internal tandem duplication of the FLT3 gene is a novel modality of elongation mutation which causes constitutive activation of the product. Leukemia. 1998;12:1333-1337.
73. Gilliland D G, Griffin J D. The roles of FLT3 in hematopoiesis and leukemia. Blood. 2002; 100:1532-1542
74. Kurzrock et al. The molecular genetics of Philadelphia chromosome-positive leukemias. New Engl J Med 1988;319:990-8.
75. O'Driscoll, M. & Jeggo, P. A. The role of double-strand break repair—insights from human genetics. Nat. Rev. Genet. 7, 45-54 (2006).
76. Chen, J. M., Cooper, D. N., Chuzhanova, N., Ferec, C. & Patrinos, G. P. Gene conversion: mechanisms, evolution and human disease. Nat. Rev. Genet. 8,762-775 (2007).
77. Chapman J R, Taylor M R G, Boulton S J. Playing the end game: DNA double-strand break repair pathway choice. Molecular Cell. 2012;47: 497-510. doi: 10.1016/j.molce1.2012.07.029.
78. Thomas K. R., Folger K. R., Capecchi M. R. High frequency targeting of genes to specific sites in the mammalian genome. Cell. 1986;44:419-428
79. Allen T. M. & Cullis P. R. Liposomal drug delivery systems: From concept to clinical applications. Adv. Drug Delivery Rev. 65,36-48 (2013).
80. T. D. Heath, J. A. Montgomery, J. R. Piper, D. Papahadjopoulos, Antibody-targeted liposomes: increase in specific toxicity of methotrexate-gamma-aspartate, Proc. Natl Acad. Sci. U.S.A. 80 (1983) 1377-1381.
81. R. Fraley, S. Subramani, P. Berg, D. Papahadjopoulos, Introduction of liposomeencapsulated SV40 DNA into cells, J. Biol. Chem. 255 (1980) 10431-10435.
82. R. Fraley, R. M. Straubinger, G. Rule, E.I. Springer, D. Papahadjopoulos, Liposomemediateddelivery of deoxyribonucleic acid to cells: enhanced efficiency of deliveryrelated to lipid composition and incubation conditions, Biochemistry 20(1981) 6978-6987.
83. R. M. Straubinger, D. Papahadjopoulos, Liposomes as carriers for intracellular delivery of nucleic acids, Methods Enzymol. 101 (1983) 512-527.
84. S. C. Semple, A. Akinc, J. Chen, A. P. Sandhu, B. L. Mui, C. K. Cho, D. W. Sah, D. Stebbing, E. J. Crosley, E. Yaworski, I. M. Hafez, J. R. Dorkin, J. Qin, K. Lam, K. G. Rajeev, K. F. Wong, L. B. Jeffs, L. Nechev, M. L. Eisenhardt, M. Jayaraman, M. Kazem, M. A. Maier, M. Srinivasulu, M. J. Weinstein, Q. Chen, R. Alvarez, S. A. Barros, S. De, S. K. Klimuk, T. Borland, V. Kosovrasti, W. L. Cantley, Y. K. Tam, M. Manoharan, M. A. Ciufolini, M. A. Tracy, A. de Fougerolles, I. MacLachlan, P. R. Cullis, T. D. Madden, M. J. Hope, Rational design of cationic lipids for siRNA delivery, Nat. Biotechnol. 28 (2010) 172-176.
85. N. Belliveau, J. Huft, P. Lin, S. Chen, A. K. K. Leung, T. J. Weaver, A. W. Wild, J. B. Lee, R. J. Taylor, Y. K. Tam, C. L. Hansen, P. R. Cullis, Microfluidic synthesis of highly potent limit-size lipid nanoparticles for in vivo delivery of siRNA., Mol. Ther. Nucleic Acids in press.
86. Horii T., Arai Y., Yamazaki M., Morita S., Kimura M., Itoh M., Abe Y. and Hatada I. (2014). Validation of microinjection methods for generating knockout mice by CRISPR/Cas-mediated genome engineering. Sci. Rep. 4,4513 10.1038/srep04513
87. Kay M A, Manno C S, Ragni M V, Larson P J, Couto L B, McClelland A, et al. Evidence for gene transfer and expression of factor IX in haemophilia B patients treated with an AAV vector. Nat Genet. 2000;24:257-61.

88.Andtbacka R H, Kaufman H L, Collichio F, et al. Talimogene laherparepvec improves durable response rate in patients with advanced melanoma. J Clin Oncol. 2015; 33(25):2780-2788.

Lengthy table referenced here
US20170218398A1-20170803-T00001
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20170218398A1). An electronic copy of the table will also be available from the USPTO upon request and payment of the fee set forth in 37 CFR 1.19(b)(3).

What is claimed is:

1. A cancer specific vector comprising:
a cancer specificity element comprising a sequence of interest as well as first and second targeting sequences,
wherein each of the first and second cancer specific targeting sequences are comprised of nucleotide sequences homologous to at least 10 bp within first and second cancer specific rearrangement sequences, respectively, and
a promoter operably linked to the sequence of interest within the cancer specificity element.

2. A cancer specific vector of claim 1, wherein at least one of the targeting sequences has 100% sequence identity to either the first or second cancer specific rearrangement sequences.

3. A cancer specific vector of claim 1, wherein at least one of the targeting sequences has at least 75% sequence identity to either the first or second cancer specific rearrangement sequences.

4. A cancer specific vector of claim 1, wherein the first and second cancer specific rearrangement sequences extend 1 MB from the chromosomal rearrangement site in either direction.

5. A cancer specific vector of claim 1, wherein the cancer specific targeting sequences are homologous to cancer specific rearrangement sequences comprising a chromosomal rearrangement site between a first mammalian chromosome and a first mammalian chromosome in a human cell.

6. A cancer specific vector of claim 1, wherein the cancer specific targeting sequences are homologous to cancer specific rearrangement sequences comprising a chromosomal rearrangement site between a first mammalian chromosome and a second mammalian chromosome, in which a second mammalian chromosome is different from a first mammalian chromosome, in a human cell.

7. A cancer specific vector of claim 5, wherein the first targeting sequence is homologous to one side of the chromosomal rearrangement site and the second targeting sequence is homologous to the opposing side of the chromosomal rearrangement site.

8. A cancer specific vector of claim 6, wherein the first targeting sequence is homologous to one side of the chromosomal rearrangement site and the second targeting sequence is homologous to the opposing side of the chromosomal rearrangement site.

9. A method of selectively inserting a sequence of interest located within a cancer specificity element of a cancer specificity vector into human cells comprising:
contacting human cells with the cancer specific vector of claim 1 under conditions sufficient for the vector to enter the cells and for the specificity element to integrate through the actions of a DNA repair pathway into a chromosomal rearrangement site (CSSM) in the genomic DNA of the human cells.

10. The method of claim 9, wherein the human cell is a human cancer cell.

11. The method of claim 9, further comprising contacting the cell with a DSB- inducing vector comprising a nucleic acid sequence that facilitates or enhances homologous recombination.

12. The method of claim 11, wherein the DSB-inducing vector comprises a meganuclease, a zinc finger nuclease (ZFN), transcription activator-like effector nuclease (TALEN), or a clustered regularly interspaced short palindromic repeats (CRISPR/Cas9) system.

13. The method of claim 9, wherein the cancer specific rearrangement sequence comprises a chromosomal breakpoint in the first mammalian chromosome in a cancer cell, and one of the targeting sequence shares sufficient sequence identity with a sequence within the cancer specific rearrangement sequence which has been translocated from a location in the first mammalian chromosome to form the breakpoint.

14. The method of claim 9, wherein the cancer specific rearrangement sequence comprises a chromosomal breakpoint in the first mammalian chromosome in a cancer cell, and one of the targeting sequence shares sequence identity with a sequence within the cancer specific rearrangement sequence which has been translocated from a second mammalian chromosome which is different than the first mammalian chromosome to form the breakpoint.

15. A method of inducing a double-strand break in a CSSM in human cells comprising:
contacting human cells with a DSB-inducing vector encoding a meganuclease, a zinc finger nuclease (ZFN), a transcription activator-like effector nuclease (TALEN), or a clustered regularly interspaced short palindromic repeats (CRISPR/Cas9) system, which upon expression, is designed to bind to any of the sequences shown in SEQ ID NOs 1-15.

16. The method of claim 15, wherein the RNA products of the DSB-inducing vector are introduced to human cells.

17. The method of claim 15, wherein the protein products of the DSB-inducing vector are introduced to human cells.

18. The method of claim 15, further comprising contacting human cells with a cancer specific vector, comprising a cancer specificity element, comprised of a sequence of interest, as well as first and second homologous targeting sequences, wherein the first and second targeting sequences are homologous to at least 10 bp of any of the sequences shown in SEQ ID NOs 1-15, under conditions sufficient for the vector to enter the cells and for the cancer specificity element to integrate through the actions of a DNA repair pathway into a chromosomal rearrangement site (CSSM) in the genomic DNA of human cells.
